(19) United States
(12) Patent Application Publication
(10) Pub. No.: US 2004/0038923 A1
(43) Pub. Date: Feb. 26, 2004

(54) Blocking inflammation by inhibiting sialylation

(76) Inventors: Jamey D. Marth, San Diego, CA (US); Lesley G. Ellies, La Jolla, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/398,520

(22) PCT Filed: Oct. 5, 2001
(86) PCT No.: PCT/US01/31403

(30) Foreign Application Priority Data
Oct. 6, 2000 (US) 60238729

(51) Int. Cl. A61K 48/00; A61K 31/715

(52) U.S. Cl. 514/44; 514/54

(57) ABSTRACT

This invention provides methods and compositions for treating and preventing inflammation. The methods for treating and preventing inflammation and related conditions involved administering to a mammal an agent that reduces activity of a sialyltransferase, such as an ST3Gal IV sialyltransferase or an ST3Gal I sialyltransferase.

### STATEMENT REGARDING FEDERALLY SPONSORED RESEARCH

This invention was made with government support under Grant No. HL57345 awarded by the National Institutes of Health. The United States Government may have certain rights in the invention.

### BACKGROUND OF THE INVENTION

1. Field of the Invention

This invention pertains to the field of treating and preventing inflammation.

Compounds and methods for modulating an inflammatory response are provided.

2. Background

A mammal often responds to cell injury, infection, or an abrupt change in a tissue by inducing an inflammatory response. Typically, an inflammatory response is initiated by endothelial cells producing molecules that attract and detain inflammatory cells (e.g., myeloid cells such as neutrophils, eosinophils, and basophils) at the site of injury. The inflammatory cells then are transported through the endothelial barrier into the surrounding tissue. The resulting accumulation of inflammatory cells, in particular neutrophils, is followed by generation of toxic oxygen particles and, release of neutrophil granules which contain acid hydrolases and degradative enzymes such as proteases, elastase, and collagenase, which contribute to local tissue breakdown and inflammation. Neutrophils can also release chemoattractants and complement activators that amplify the inflammation.

Although the inflammatory response can play a role in the healing process by destroying, diluting, and isolating injurious agents and stimulating repair of the affected tissue, inflammatory responses can also be harmful, and indeed life-threatening. Five symptoms often characterize the inflammatory response: pain, redness, heat, swelling, and loss of function. For example, inflammation results in leakage of plasma from the blood vessels. Although this leakage can have beneficial effects, it causes pain and when uncontrolled can lead to loss of function and death (such as adult respiratory distress syndrome). Anaphylactic shock, arthritis, and gout are among the conditions that are characterized by uncontrolled or inappropriate inflammation.

Inflammatory responses differ from immune responses mediated by T- and B-lymphocytes in that an inflammatory response is non-specific. While antibodies and MHC-mediated immune responses are specific to a particular pathogen or other agent, the inflammatory response does not involve identification of a specific agent. Both inflammatory responses and specific immune responses, however, involve extravasation of the respective cell types from the blood vessels to the site of tissue injury or infection. Moreover, several of the receptors that mediate extravasation of lymphocytes are also involved in extravasation of inflammatory cells. In particular, lymphocyte trafficking to lymph nodes under normal circumstances is mediated by selectins that are expressed by cells of the vascular endothelium in response to cytokine induction. Selectins are also involved in the recruitment of neutrophils to the vascular endothelium during inflammation (reviewed in Kansas (1996) Blood 88: 3259-87; McEver and Cummings (1997) J. Clin. Invest. 100: 485-91). Three types of selectins are involved in the interaction between leukocytes and the vascular endothelium. E-selectin (also called endothelial-leukocyte adhesion molecule-1, ELAM-1) and P-selectin are expressed on activated endothelium. P-selectin is also present on activated platelets, while L-selectin is found on lymphocytes. Selectin deficiencies result in varying degrees of impaired lymphocyte trafficking, reduced neutrophil recruitment to sites of inflammation and decreased leukocyte turnover (Arbones et al. (1994) Immunity 1: 247-260; Johnson et al. (1995) Blood 86: 1106-14; Labow et al. (1995) Immunity 1: 709-720; Mayadas et al. (1993) Cell 74: 541-554).

Binding of leukocytes to selectins is at least partially mediated by oligosaccharide ligands that are displayed on the surface of the leukocytes. The oligosaccharide ligands are generally attached to glycoproteins and glycolipids. The types of oligosaccharides that carry the physiologically relevant selectin ligands are largely undefined at present, with a variety of possibilities existing among N-glycans, O-glycans, glycolipids, as well as proteoglycans (reviewed in Varki (1997) J. Clin. Invest. 99: 158-162).

Leukocyte binding to selectins is followed by additional steps. For example, the transient binding of leukocyte ligands to selectins results in “rolling” or “tethering” in which the leukocytes roll along the surface of the endothelial cells. The leukocytes then receive signals that activate leukocyte integrins into a high affinity state, which results in the leukocytes becoming more firmly bound to the endothelial wall of the blood vessel. Leukocyte extravasation then occurs, a process in which the leukocytes pass through the endothelial wall and enter the underlying tissue. However, these steps in the inflammation process which are downstream of the initial selectin binding previously were not well understood. This has hampered the ability to develop treatments that are effective against chronic and otherwise undesirable inflammation. The present invention fulfills this and other needs.

### SUMMARY OF THE INVENTION

The present invention provides novel prophylactic, treatment and diagnostic methods for inflammation and related conditions.

In some embodiments, the invention provides methods for modulating the amount of cell-surface oligosaccharides that comprise a terminal α2,3-linked sialic acid that are attached to inflammatory cells in an animal. The methods can involve administering to the animal an agent that causes a decrease in α2,3-sialyltransferase activity in the animal. For example, the methods can involve reducing the amount of ST3Gal IV or ST3Gal I sialyltransferase activity in the animal. The decrease in α2,3-sialyltransferase activity can be achieved by administering an agent that decreases expression of a gene that encodes an α2,3-sialyltransferase, and/or by administering an agent that inhibits enzymatic activity of an α2,3-sialyltransferase polypeptide. Alternatively, the reduction in the amount of cell-surface oligosaccharides that comprise a terminal α2,3-linked sialic acid can be achieved by administering an agent that reduces the amount of acceptor oligosaccharide that is available for sialylation by an α2,3-sialyltransferase.

Also provided by the invention are methods for monitoring the efficacy of a method for inhibiting α2,3-sialyltransferase in a mammal. These methods involve testing cells obtained from the mammal for the presence or absence of a cell-surface oligosaccharide having a terminal α2,3-linked sialic acid, wherein the absence of the terminal α2,3-linked sialic acid is indicative of inhibition of α2,3-sialyltransferase activity.

In another embodiment, the invention provides eukaryotic cells in which a non-naturally occurring mutation is present in an ST3Gal IV or ST3Gal I allele. At least one, and sometimes two or more alleles have a mutation. In presently preferred embodiments, the mutation either disrupts the expression of ST3 Gal IV or ST3 Gal I or results in expression of an ST3Gal IV or ST3Gal I polypeptide that has reduced activity compared to an ST3Gal IV or ST3Gal I polypeptide encoded by a gene that lacks the mutation.

The invention also provides transgenic and chimeric animals that have non-naturally occurring mutation in an ST3Gal IV or ST3Gal I allele in at least some of the cells of the animal.

### BRIEF DESCRIPTION OF THE DRAWINGS

FIGS. 1A-C show a strategy employed to disrupt the ST3Gal-IV gene and introduce the disrupted gene in embryonic stem cells, from which knockout mice were obtained. FIG. 1A: The wild type ST3Gal-IV genomic locus was used in conjunction with the pflox vector to construct a targeting vector in which exons containing the large sialyl motif were flanked by loxP sites (ST3Gal-IV^{F[tkneo]}). Restriction enzyme sites indicated are Bam HI (B) Avr II (A), Eco RI (E), Hind III (H), Kpn I (K), Not I (N), Sal I (Sa), Spe I (Sp) and Xba I (X). FIG. 1B: Transient Cre expression in ST3Gal-IV-targeted ES cells resulted in subclones isolated that carry a ST3Gal-IV^{-/-} (systemic-null) or ST3Gal-IV^{F} (conditional-null) mutation. FIG. 1C: Southern blot analysis of a Avr II/Spe I digest of ES cell DNA probed with a loxP probe confirmed the expected structures. Wild type R1 ES cell DNA did not hybridize to the loxP probe. Three loxP sites are present in a targeted parental clone (21-6), one loxP site is present in each of two ST3Gal-IV^{-/-} subclones (21-F1 and 21-D1) and two loxP sites are present in the ST3Gal-IV^{F} subclones (21-A3 and 21-E1). FIG. 1D shows hybridization of the genomic probe shown in FIG. 1A to Hind III-digested DNA obtained from the tail of progeny from a heterozygous mating of a ST3Gal-IV^{-/-} chimera. Both the 6.8 kb wild type allele and the 5.3 kb mutant allele were visible in heterozygous (+/−) progeny, while only the 6.8 kb wild-type allele was found in the homozygous normal (+/+) animal, and only the 5.3 kb mutant allele was observed in the homozygous ST3Gal-IV deficient (−/−) mouse.

FIGS. 2A-C show ST3Gal-IV expression in various tissues of the knockout mice, as well as an analysis of the oligosaccharide structures present on myeloid cells obtained from homozygous normal (+/+), heterozygous (+/−), and homozygous ST3Gal-IV deficient (−/−) mice. FIG. 2A: Total RNA (lower panel) from various tissues obtained from a normal mouse was hybridized to a probe specific for ST3Gal-IV (upper panel). FIG. 2B: RNA from the small intestine and colon of wild-type and ST3Gal-IV^{-/-} mice were hybridized to a labeled full-length mouse ST3Gal-IV cDNA. FIG. 2C: Myeloid cells of the bone marrow were double-stained with monoclonal antibodies that recognize myeloid cells (CD11b) and the lectin chimeras, siglec 1, E-selectin and P-selectin, as well as the PNA and ECA lectins and an antibody that recognizes the CD43 130 kD (1B11) and subjected to flow cytometric analysis. Myeloid cells were detected by an anti-CD11b antibody.

FIG. 3 shows the loss of Gr-1⁺ neutrophil recruitment during inflammation. Following a thioglycollate injection into the peritoneal cavity, neutrophil recruitment is analyzed at various times post-injection. Control values are shown (WT) and a loss of Gr-1+ cell recruitment in ST3Gal-IV-deficient mice is evident at 24 hours. Some recruitment of Gr-1⁺ cells is seen at 24 hours, and represents the macrophage population.

FIG. 4 shows the levels of E- and P-selectin ligands on Gr-1+ myeloid cells. Greatly reduced levels of E- and P-selectin binding are evident on Core 2 GlcNAcT-deficient Gr-1⁺ cells (light line), and close to background binding levels as observed on wild-type cells in the presence of EDTA (dotted line). In comparison to wild-type Gr-1+ cells (black line) a partial reduction in E- and P-selectin binding is only observed in ST3Gal-IV-deficient Gr-1+ cells. Data are representative of 4 separate experiments.

FIGS. 5A-5C is a schematic diagram of the ST3Gal I gene structure and the construction of ST3Gal I mutants. FIG. 5A shows the structure of the wild-type mouse ST3Gal I gene as found on genomic clone 129 Sv/J, and the pflox construct that was used to make a targeting vector as shown in FIG. 5B. Upon homologous recombination with the ST3Gal I^{wt} locus in mouse ES cells, as shown in FIG. 5B, ES cells heterozygous for the ST3Gal I^{F[tkneo]} construct were obtained. Cre-mediated recombination with ganciclovir selection resulted in two types of deletions as shown in FIG. 5C, the ST3Gal I⁻ deletion, which resulted from a Type 1 deletion lacks exon 2 of the ST3 Gal I gene, and the ST3 Gal I^{F} construct which resulted from a Type 2 deletion.

### DETAILED DESCRIPTION

Definitions

The following abbreviations are used herein:

| | |
|---|---|
| Ara = | arabinosyl; |
| Fru = | fructosyl; |
| Fuc = | fucosyl; |
| Gal = | galactosyl; |
| GalNAc = | N-acetylgalactosaminyl; |
| Glc = | glucosyl; |
| GlcNAc = | N-acetylglucosaminyl; |
| Man = | mannosyl; and |
| NeuAc = | sialyl (N-acetylneuraminyl). |

Oligosaccharides are considered to have a reducing end and a non-reducing end, whether or not the saccharide at the reducing end is in fact a reducing sugar. In accordance with accepted nomenclature, oligosaccharides are depicted herein with the non-reducing end on the left and the reducing end on the right.

All oligosaccharides described herein are described with the name or abbreviation for the non-reducing saccharide (e.g., Gal), followed by the configuration of the glycosidic bond (α or β), the ring bond, the ring position of the reducing saccharide involved in the bond, and then the name or abbreviation of the reducing saccharide (e.g., GlcNAc). The linkage between two sugars may be expressed, for example, as 2,3,2→3, or (2,3). Each saccharide is a pyranose.

The term “sialic acid” refers to any member of a family of nine-carbon carboxylated sugars. The most common member of the sialic acid family is N-acetyl-neuraminic acid (2-keto-5-acetamindo-3,5-dideoxy-D-glycero-D-galactononulopyranos-1-onic acid (often abbreviated as Neu5Ac, NeuAc, or NANA). A second member of the family is N-glycolyl-neuraminic acid (Neu5Gc or NeuGc), in which the N-acetyl group of NeuAc is hydroxylated. A third sialic acid family member is 2-keto-3-deoxy-nonulosonic acid (KDN) (Nadano et al. (1986) J. Biol. Chem. 261: 11550-11557; Kanamori et al. (1990) J. Biol. Chem. 265: 21811-21819. Also included are 9-substituted sialic acids such as a 9-O-C1-C6 acyl-Neu5Ac like 9-O-lactyl-Neu5Ac or 9-O-acetyl-Neu5Ac, 9deoxy-9-fluoro-Neu5Ac and 9-azido-9-deoxy-Neu5Ac. For review of the sialic acid family, see, e.g., Varki (1992) Glycobiology 2: 25-40; Sialic Acids: Chemistry, Metabolism and Function, R. Schauer, Ed. (Springer-Verlag, New York (1992). The synthesis and use of sialic acid compounds in a sialylation procedure is disclosed in international application WO 92/16640, published Oct. 1, 1992.

Much of the nomenclature and general laboratory procedures required in this application can be found in Sambrook, et al. (1989) Molecular Cloning: A Laboratory Manual (2nd Ed.), Vol. 1-3, Cold Spring Harbor Laboratory, Cold Spring Harbor, N.Y. . The manual is hereinafter referred to as “Sambrook et al.”

The term “nucleic acid” refers to a deoxyribonucleotide or ribonucleotide polymer in either single- or double-stranded form, and unless otherwise limited, encompasses known analogues of natural nucleotides that hybridize to nucleic acids in manner similar to naturally occurring nucleotides. Unless otherwise indicated, a particular nucleic acid sequence includes the complementary sequence thereof.

An “inhibitory nucleic acid” is any nucleic acid or modified nucleic acid used or designed for use in inhibitory nucleic acid therapy. “Inhibitory nucleic acid therapy” refers to the use of inhibitory nucleic acids to inhibit gene expression, for example, inhibition of DNA transcription, inhibition of RNA processing, transport or translation, or inhibition of protein synthesis. Inhibitory nucleic acid therapy includes the variety of approaches for treatment of disease using nucleic acids or modified nucleic acids as described herein. Various inhibitory nucleic acid therapies are discussed in detail below.

The term “operably linked” refers to functional linkage between a nucleic acid expression control sequence (such as a promoter, signal sequence, or array of transcription factor binding sites) and a second nucleic acid sequence, wherein the expression control sequence affects transcription and/or translation of the nucleic acid corresponding to the second sequence.

The term “recombinant” when used with reference to a cell indicates that the cell replicates a heterologous nucleic acid, or expresses a peptide or protein encoded by a heterologous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques.

A “heterologous sequence” or a “heterologous nucleic acid”, as used herein, is one that originates from a source foreign to the particular host cell, or, if from the same source, is modified from its original form.

A “subsequence” refers to a sequence of nucleic acids or amino acids that comprise a part of a longer sequence of nucleic acids or amino acids (e.g., polypeptide) respectively.

A “recombinant expression cassette” or simply an “expression cassette” is a nucleic acid construct, generated recombinantly or synthetically, with nucleic acid elements that are capable of affecting expression of a structural gene in hosts compatible with such sequences. Expression cassettes include at least promoters and optionally, transcription termination signals. Typically, the recombinant expression cassette includes a nucleic acid to be transcribed (e.g., a nucleic acid encoding a desired polypeptide), and a promoter. Additional factors necessary or helpful in effecting expression may also be used as described herein. For example, an expression cassette can also include nucleotide sequences that encode a signal sequence that directs secretion of an expressed protein from the host cell. Transcription termination signals, enhancers, and other nucleic acid sequences that influence gene expression, can also be included in an expression cassette.

The term “isolated” is meant to refer to material which is substantially or essentially free from components which normally accompany the enzyme as found in its native state. Thus, the enzymes of the invention do not include materials normally associated with their in situ environment. Typically, isolated proteins of the invention are at least about 80% pure, usually at least about 90%, and preferably at least about 95% pure as measured by band intensity on a silver stained gel or other method for determining purity. Protein purity or homogeneity can be indicated by a number of means well known in the art, such as polyacrylamide gel electrophoresis of a protein sample, followed by visualization upon staining. For certain purposes high resolution will be needed and HPLC or a similar means for purification utilized.

The phrase “substantially identical,” in the context of two nucleic acids or polypeptides, refers to two or more sequences or subsequences that have at least 70%, preferably 80%, most preferably 90-95% nucleotide or amino acid residue identity, when compared and aligned for maximum correspondence, as measured using one of the following sequence comparison algorithms or by visual inspection. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman (1981) Adv. Appl. Math. 2: 482, by the homology alignment algorithm of Needleman & Wunsch (1970) J. Mol. Biol. 48: 443, by the search for similarity method of Pearson & Lipman (1988) Proc. Nat'l. Acad. Sci. USA 85: 2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra).

Another example of algorithm that is suitable for determining percent sequence identity and sequence similarity is the BLAST algorithm, which is described in Altschul et al. (1990) J. Mol. Biol. 215: 403410. Software for performing BLAST analyses is publicly available through the National Center for Biotechnology Information http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N-4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89: 10915).

“Conservatively modified variations” of a particular polynucleotide sequence refers to those polynucleotides that encode identical or essentially identical amino acid sequences, or where the polynucleotide does not encode an amino acid sequence, to essentially identical sequences. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified by a codon, the codon can be altered to any of the corresponding codons described without altering the encoded polypeptide. Such nucleic acid variations are “silent variations,” which are one species of “conservatively modified variations.” Every polynucleotide sequence described herein which encodes a polypeptide also describes every possible silent variation, except where otherwise noted. One of skill will recognize that each codon in a nucleic acid (except AUG, which is ordinarily the only codon for methionine) can be modified to yield a functionally identical molecule by standard techniques. Accordingly, each “silent variation” of a nucleic acid which encodes a polypeptide is implicit in each described sequence.

Furthermore, one of skill will recognize that individual substitutions, deletions or additions which alter, add or delete a single amino acid or a small percentage of amino acids (typically less than 5%, more typically less than 1%) in an encoded sequence are “conservatively modified variations” where the alterations result in the substitution of an amino acid with a chemically similar amino acid. Conservative substitution tables providing functionally similar amino acids are well known in the art. See, e.g., Creighton (1984) Proteins, W. H. Freeman and Company. In addition, individual substitutions, deletions or additions which alter, add or delete a single amino acid or a small percentage of amino acids in an encoded sequence are also “conservatively modified variations”.

The term “transgenic” refers to a cell that includes a specific genetic modification that was introduced into the cell, or an ancestor of the cell. Such modifications can include one or more point mutations, deletions, insertions, or combinations thereof. When referring to an animal, the term “transgenic” means that the animal includes cells that are transgenic, and descendants of such animals. An animal that is composed of both transgenic and non-transgenic cells is referred to herein as a “chimeric” animal.

Description of the Preferred Embodiments

The present invention provides compositions and methods for reducing and/or preventing inflammation in a mammal. The invention is based on the discovery that ablation of one or more alleles of the ST3Ga IV or ST3Gal I sialyltransferase results in a decrease in neutrophil recruitment and extravasation in response to an inflammatory stimulus.

Accordingly, the compositions and methods of the invention are useful for treating and/or preventing inflammation by causing a decrease in the amount of cell-surface oligosaccharides that display a terminal α2,3-linked sialic acid, which cell-surface oligosaccharides are attached to a cell involved in inflammation.

Prophylactic and Therapeutic Methods for Inflammation

In some embodiments, the invention provides methods for reducing inflammation by decreasing the amount of cell surface N-linked and/or O-linked oligosaccharides that terminate in an α2,3-linked sialic acid.

A. Reducing Biosynthesis of Oligosaccharides Having a Terminal α2,3-Linked Sialic Acids

The invention provides several methods by which reductions in biosynthesis of oligosaccharides that terminate in an α2,3-linked sialic acid can be accomplished. The expression of an α2,3 sialyltransferase gene can be inhibited, for example, or the enzymatic activity of the protein can be inhibited. Alternatively, the oligosaccharide that serves as an acceptor for the α2,3 sialyltransferase-catalyzed reaction can be modified, e.g., by addition or removal of a saccharide residue from the acceptor to render the oligosaccharide no longer an acceptable acceptor for the sialyltransferase.

Sialyltransferase Inhibitors

In some embodiments, reductions in inflammation are obtained by inhibiting the enzymatic activity of an α2,3-sialyltransferase, such as ST3Gal IV or ST3Gal I. Enzyme inhibition generally involves the interaction of a substance with an enzyme so as to decrease the rate of the reaction catalyzed by that enzyme.

Several inhibitors of sialyltransferases are known in the art. For example, analogs of sialyltransferase substrates are suitable for use as inhibitors. Analogs of both the donor (e.g., analogs of CMP-sialic acid) and the acceptor have been reported which serve as sialyltransferase inhibitors (Schaub et al. (1998) Glycoconjugate J. 15: 345-354; Schaub and Schmidt (1996) Abstract C 10, Second European Conference on Carbohydrate Mimics, La Garda (Italy); Amann et al. (1998) Chem. Eur. J. 4: 1106-1115; Miller et al. (1998) Tetrahedron Lett. 39: 509-512; Korytnyk et al. (1980) Eur. J. Med. Chem. 15: 77-84; Kijima-Suda et al. (1986) Cancer Res. 46: 858-862; Khan et al. (1992) In Glycoconjugates, Composition, Structure, Function (Eds.: H. J. Allen, E. C. Kisailus). M. Dekker, New York, pp. 361-378 and references therein; Hashimoto et al. (1993) Carbohydr. Res. 247: 179-193; Imamoto and Hashimoto (1996) Tetrahedron Lett. 37: 1451-1454; Kleineidam et al. (1997) Glycoconjugate J. 14: 57-66). Transition state analogs are also useful as sialyltransferase inhibitors (Schaub et al., supra., Schaub and Schmidt, supra.; Amann et al., supra., and WO 008040). Other sialyltransferase inhibitors are described in Cambron and Leskawa (1993) Biochem. Biophys. Res. Commun. 193:585-90.

α2,3-sialyltransferase activity can also be regulated by modulation of the phosphorylation state of the enzyme. Phosphorylation of a serine residue in ST3Gal IV by, for example, protein kinase A or C results in a decrease in sialyltransferase activity (Gu et al. (1995) J. Neurochem. 64:2295-302). Activity can be restored by treatment with a phosphatase. Protein kinase activators and phosphatase inhibitors can therefore be administered to reduce ST3Gal IV activity. One example of a suitable protein kinase inhibitor is a subtype of the 14-3-3 protein family that has been shown to be associated with ST3Gal IV (Gao et al. (1996) Biochem. Biophys. Res. Commun. 224:103-7). Other examples of suitable protein kinase activators and phosphatase inhibitors are described in Bieberich et al. (1998) J. Neurochem. 71:972-9.

Additional inhibitors of the α2,3-sialyltransferase can be readily identified by screening methods known to those of skill in the art. Sialyltransferase activity and its inhibition is typically assayed according to standard methods for determining enzyme activity. For a general discussion of enzyme assays, see, Rossomando, “Measurement of Enzyme Activity” in Guide to Protein Purification, Vol. 182, Methods in Enzymology (Deutscher ed., 1990), and Fersht, Enzyme Structure and Mechanism (2d ed. 1985). Enzyme inhibition of kinetically complex systems involving more than one substrate, as is the case for glycosyltransferases, are described in Segel, Enzyme Kinetics, (Wiley, N.Y. 1975), which is incorporated herein by reference.

An assay for α2,3-sialyltransferase activity typically contains a buffered solution adjusted to physiological pH, a source of divalent cations, a donor substrate (usually labeled CMP-sialic acid), an acceptor substrate (e.g., Galβ1,4GlcNAc or Galβ1,3GalNAc), the sialyltransferase, and the compound whose inhibitory activity is to be tested. After a predetermined time, typically at 23° C. or 37° C., the reaction is stopped and the sialylated product is isolated and measured according to standard methods (e.g., in a scintillation counter). Sialyltransferase assays which use a UV-labeled acceptor and lead to a UV-labeled product that can be readily separated by reverse phase HPLC and quantitated by UV spectroscopy are described in Schaub et al. (1998) Glycoconjugate J. 15: 345-354. See also, Kajihara et al. (1994) Carbohydr. Res. 264, C1-C5; (1995) J. Org. Chem. 60: 5732-5735. Inhibition of sialyltransferase activity in an assay as defined herein refers to a decrease in enzyme specific activity in the presence of an inhibitory agent of at least about 50%, more preferably at least about 70%, and still more preferably at least about 90%, compared to the activity in the absence of the agent.

Screening can be employed to identify α2,3-sialyltransferase inhibitors that are present in a mixture of synthetically produced compounds or alternatively in a naturally occurring mixture, such as a cell culture broth. Suitable cells include any cultured cells such as mammalian, insect, microbial or plant cells. Microbial cell cultures are composed of any microscopic organism such as bacteria, protozoa, yeast, fungi and the like. In the typical screening assay, a sample, such as a fungal broth, is added to a standard sialyltransferase assay. If inhibition of activity as compared to control assays is found, the mixture is usually fractionated to identify components of the sample that provide the inhibiting activity. The sample is fractionated using standard methods such as ion exchange chromatography, affinity chromatography, electrophoresis, ultrafiltration, HPLC and the like. See, e.g., Protein Purification, Principles and Practice, (Springer-Verlag, 1982). Each isolated fraction is then tested for inhibitory activity. If desired, the fractions are then further subfractionated and tested. This subfractionation and testing procedure can be repeated as many times as desired.

By combining various standard purification methods, a substantially pure compound suitable for in vivo therapeutic testing can be obtained. A substantially pure blocking agent as defined herein is an inhibitory compound which migrates largely as a single band under standard electrophoretic conditions or largely as a single peak when monitored on a chromatographic column. More specifically, compositions of substantially pure blocking agents will comprise less than ten percent miscellaneous compounds.

Inhibitors can be classified according a number of criteria. For example, they may be reversible or irreversible. An irreversible inhibitor dissociates very slowly, if at all, from its target enzyme because it becomes very tightly bound to the enzyme, either covalently or noncovalently. Reversible inhibition, in contrast, involves an enzyme-inhibitor complex which may dissociate. Inhibitors can also be classified according to whether they are competitive, noncompetitive or uncompetitive inhibitors. In competitive inhibition for kinetically simple systems involving a single substrate, the enzyme can bind either the substrate or the inhibitor, but not both. Typically, competitive inhibitors resemble the substrate or the product(s) and bind the active site of the enzyme, thus blocking the substrate from binding the active site. A competitive inhibitor diminishes the rate of catalysis by effectively reducing the affinity of the substrate for the enzyme. Typically, an enzyme may be competitively inhibited by its own product because of equilibrium considerations. Since the enzyme is a catalyst, it is in principle capable of accelerating a reaction in the forward or reverse direction. Noncompetitive inhibitors allow the enzyme to bind the substrate at the same time it binds the inhibitor. A noncompetitive inhibitor acts by decreasing the turnover number of an enzyme rather than diminishing the proportion of free enzyme. Another possible category of inhibition is mixed or uncompetitive inhibition, in which the inhibitor affects the binding site and also alters the turnover number of the enzyme.

### Inhibition of α2,3-sialyltransferase Gene Expression

Inhibition of ST3Gal IV gene expression can also be achieved through the use of inhibitory nucleic acids. Inhibitory nucleic acids can be single-stranded nucleic acids that are complementary to, and thus can specifically hybridize to, a target sequence such as a nucleic acid that encodes ST3 Gal IV. By binding to the appropriate target sequence, an RNA-RNA, a DNA-DNA, or RNA-DNA duplex or triplex is formed. These nucleic acids are often termed “antisense” because they are usually complementary to the sense or coding strand of the gene, although recently approaches for use of “sense” nucleic acids have also been developed. The term “inhibitory nucleic acids” as used herein, refers to both “sense” and “antisense” nucleic acids.

In one embodiment, the inhibitory nucleic acid can specifically bind to a target nucleic acid that encodes an α2,3-sialyltransferase sialyltransferase. The nucleotide sequence of a human ST3Gal IV cDNA is reported in Kitagawa and Paulson (1994) J. Biol. Chem. 269: 1394-401. This nucleotide can be used as a probe for the identification of α2,3-sialyltransferase-encoding nucleic acids from other species. From the human or other α2,3-sialyltransferase-encoding nucleotide sequences, one can derive suitable inhibitory nucleic acids. Administration of such inhibitory nucleic acids to a mammal can reduce inflammation by reducing or eliminating the biosynthesis of Siaα2,3Gal-containing oligosaccharides.

By binding to the target nucleic acid, the inhibitory nucleic acid can inhibit the function of the target nucleic acid. This could, for example, be a result of blocking DNA transcription, processing or poly(A) addition to mRNA, DNA replication, translation, or promoting inhibitory mechanisms of the cells, such as promoting RNA degradation. Inhibitory nucleic acid methods therefore encompass a number of different approaches to altering expression of specific genes that operate by different mechanisms. These different types of inhibitory nucleic acid technology are described in Helene and Toulme (1990) Biochim. Biophys. Acta. 1049: 99-125.

Inhibitory nucleic acid therapy approaches can be classified into those that target DNA sequences, those that target RNA sequences (including pre-mRNA and mRNA), those that target proteins (sense strand approaches), and those that cause cleavage or chemical modification of the target nucleic acids.

Approaches targeting DNA fall into several categories. Nucleic acids can be designed to bind to the major groove of the duplex DNA to form a triple helical or “triplex” structure. Alternatively, inhibitory nucleic acids are designed to bind to regions of single stranded DNA resulting from the opening of the duplex DNA during replication or transcription. See Helene and Toulme, supra.

More commonly, inhibitory nucleic acids are designed to bind to mRNA or mRNA precursors. Inhibitory nucleic acids are used to prevent maturation of pre-mRNA. Inhibitory nucleic acids may be designed to interfere with RNA processing, splicing or translation. The inhibitory nucleic acids are often targeted to mRNA. In this approach, the inhibitory nucleic acids are designed to specifically block translation of the encoded protein. Using this approach, the inhibitory nucleic acid can be used to selectively suppress certain cellular functions by inhibition of translation of mRNA encoding critical proteins. For example, an inhibitory antisense nucleic acid complementary to regions of a target mRNA inhibits protein expression. See, e.g., Wickstrom E. L. et al. (1988) Proc. Nat'l. Acad. Sci. USA 85:1028-1032 and Harel-Bellan et al. (1988) Exp. Med., 168:2309-2318. As described in Helene and Toulme, inhibitory nucleic acids targeting mRNA have been shown to work by several different mechanisms in order to inhibit translation of the encoded protein(s).

The inhibitory nucleic acids introduced into the cell can also encompass the “sense” strand of the gene or mRNA to trap or compete for the enzymes or binding proteins involved in mRNA translation. See Helene and Toulme.

Lastly, the inhibitory nucleic acids can be used to induce chemical inactivation or cleavage of the target genes or mRNA. Chemical inactivation can occur by the induction of crosslinks between the inhibitory nucleic acid and the target nucleic acid within the cell. Alternatively, irreversible photochemical reactions can be induced in the target nucleic acid by means of a photoactive group attached to the inhibitory nucleic acid. Other chemical modifications of the target nucleic acids induced by appropriately derivatized inhibitory nucleic acids may also be used.

Cleavage, and therefore inactivation, of the target nucleic acids may be effected by attaching a substituent to the inhibitory nucleic acid which can be activated to induce cleavage reactions. The substituent can be one that effects either chemical, photochemical or enzymatic cleavage. Alternatively cleavage can be induced by the use of ribozymes or catalytic RNA. In this approach, the inhibitory nucleic acids would comprise either naturally occurring RNA (ribozymes) or synthetic nucleic acids with catalytic activity.

Once α2,3-sialyltransferase inhibitors are identified, they can be tested for ability to reduce inflammation upon administration to laboratory animals. Animals can be treated with pharmacological doses of the inhibitor to block addition of α2,3-linked sialic acid to cell surface carbohydrates of cells involved in inflammation. The effect of the inhibitor on inflammation is then determined.

### Reduction in Suitable Acceptors for Sialyltransferase

Another approach to reducing the amount of cell-surface oligosaccharides that terminate in an α2,3-linked sialic acid and are displayed on inflammatory cells is to reduce the amount of acceptor that is available for sialylation by an α2,3-sialyltransferase. Methods for reducing the amount of acceptor are described in, for example, WO98/54365.

B. Administration of Anti-inflammatory Agents

The invention provides methods and compositions for treating and preventing inflammation. In therapeutic applications, the α2,3-sialyltransferase inhibitors of the invention are administered to an individual already suffering from inflammation. Compositions that contain the inhibitors are administered to a patient in an amount sufficient to decrease the amount of α2,3-sialyltransferase activity, and to cure or at least partially arrest the symptoms and/or complications of the inflammation. For example, the α2,3-sialyltransferase inhibitors can arrest the further development of the inflammation. An amount adequate to accomplish this is defined as “therapeutically effective dose.” Amounts effective for this use will depend on, e.g., the inhibitor composition, the manner of administration, the stage and severity of the disease being treated, the weight and general state of health of the patient, and the judgment of the prescribing physician. Therapeutic administration can begin at the first sign of disease or the detection or shortly after diagnosis in the case of inflammation. This is often followed by repeated administration until at least symptoms are substantially abated and for a period thereafter.

Therapeutically effective amounts of the α2,3-sialyltransferase inhibitor compositions of the present invention generally range, for the initial administration (that is for therapeutic or prophylactic administration), from about 1.0 mg to about 10 g of ST3Gal IV inhibitor for a 70 kg patient, usually from about 10 mg to about 5 g, and preferably between about 2 mg and about 1 g. These doses can be followed by repeated administrations over weeks to months depending upon the patient's response and condition by measuring immune system activity.

For prophylactic use, administration should be given to individuals that fall into groups that are at risk for developing inflammation. A “prophylactic dose” is that which is effective to maintain the concentration of α2,3-sialylated oligosaccharides at a desired level that is associated with reduced risk of atherosclerosis.

The pharmaceutical compositions for therapeutic or prophylactic treatment are intended for parenteral, topical, oral or local administration. Typically, the pharmaceutical compositions are administered parenterally, e.g., intravenously, subcutaneously, intradermally, or intramuscularly. Compositions of the invention are also suitable for oral administration. Thus, the invention provides compositions for parenteral administration which comprise a solution of the glycosyltransferase inhibiting agent dissolved or suspended in an acceptable carrier, preferably an aqueous carrier. A variety of aqueous carriers may be used, e.g., water, buffered water, 0.9% saline, 0.3% glycine, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

The concentration of α2,3-sialyltransferase inhibiting agents of the invention in the pharmaceutical formulations can vary widely, i.e., from less than about 0.1%, usually at or at least about 2% to as much as 20% to 50% or more by weight, and will be selected primarily by fluid volumes, viscosities, etc., in accordance with the particular mode of administration selected.

The α2,3-sialyltransferase inhibitors of the invention can also be administered via liposomes, which serve to target the conjugates to a particular tissue, such as myeloid tissue, as well as increase the half-life of the peptide composition. Liposomes include emulsions, foams, micelles, insoluble monolayers, liquid crystals, phospholipid dispersions, lamellar layers and the like. In these preparations the inhibitor to be delivered is incorporated as part of a liposome, alone or in conjunction with a molecule which binds to, e.g., a receptor prevalent among myeloid cells, or with other therapeutic or immunogenic compositions. Thus, liposomes filled with a desired inhibitor of the invention can be directed to the site of myeloid cells, where the liposomes then deliver the selected inhibitor compositions. Liposomes for use in the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al. (1980) Ann. Rev. Biophys. Bioeng. 9: 467, U.S. Pat. Nos. 4,235,871, 4,501,728 and 4,837,028.

The targeting of liposomes using a variety of targeting agents is well known in the art (see, e.g., U.S. Pat. Nos. 4,957,773 and 4,603,044). For targeting to the immune cells, a ligand to be incorporated into the liposome can include, e.g., antibodies or fragments thereof specific for cell surface determinants of the desired target cells. A liposome suspension containing a peptide or conjugate can be administered intravenously, locally, topically, etc. in a dose which varies according to, inter alia, the manner of administration, the conjugate being delivered, and the stage of the disease being treated.

For solid compositions, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more conjugates of the invention, and more preferably at a concentration of 25%-75%.

For aerosol administration, the inhibitors are preferably supplied in a suitable form along with a surfactant and propellant. Typical percentages of α2,3-sialyltransferase inhibitors are 0.01%-20% by weight, preferably 1%-10%. The surfactant must, of course, be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides can be employed. The surfactant can constitute 0.1%-20% by weight of the composition, preferably 0.25-5%. The balance of the composition is ordinarily propellant. A carrier can also be included, as desired, as with, e.g., lecithin for intranasal delivery.

Alternatively, DNA or RNA that inhibits expression of one or more glycosyltransferase inhibitors, such as an antisense nucleic acid or a nucleic acid that encodes a peptide that blocks expression or activity of α2,3-sialyltransferase can be introduced into patients to achieve inhibition. U.S. Pat. No. 5,580,859 describes the use of injection of naked nucleic acids into cells to obtain expression of the genes which the nucleic acids encode.

During the course of treatment, inflammation is preferably monitored and the frequency and amounts of inhibitor administration are adjusted as required.

Diagnostic Methods

The present invention also provides methods of determining the degree of α2,3-sialylation by detecting the levels of α2,3 sialylgalactosides in a sample from a patient. The diagnostic methods are also useful for monitoring the effectiveness of a prophylactic or treatment regime for atherosclerosis-related conditions, for example. Samples that are suitable for use in the diagnostic methods of the invention include, for example, myeloid cells and other blood cells.

The methods involve contacting a sample from a patient or other animal with a detection moiety that binds to a particular oligosaccharide structure, e.g., an α2,3-sialylgalactoside. Standard methods for detection of desired carbohydrate structures are known. For instance, specific lectins or antibodies raised against oligosaccharide can be used. For example, members of the siglec family of lectins that bind to oligosaccharides that are terminated with α2,3-linked sialic acid are suitable. For example, the MAL II lectin, which can be isolated from Maackia amurensis seeds, is suitable.

Alternatively, rather than using a binding moiety that binds to the sialic acid-terminated oligosaccharides, one can employ a binding moiety that binds to the acceptor for the ST3Gal IV and/or ST3Gal I. In the absence of a particular sialyltransferase, the concentration of acceptor moieties tends to increase. Thus, decreased levels of ST3Gal IV and/or ST3Gal I activity will result in an increase in the concentration of such unsialylated acceptor moieties. For example, one can employ a lectin, antibody, or other moiety that binds to unsialylated Galβ1,4GlcNAc or Galβ1,3GalNAc. Lectins that are suitable for this purpose include, for example, peanut agglutinin (PNA) or Erythrina cristagalli (ECA) lectin.

Glycosyltransferases themselves, in particular the acceptor binding domain of a glycosyltransferase, are also useful as binding moieties in the diagnostic assays of the invention. A deficiency of ST3Gal IV and/or ST3Gal I sialyltransferase causes a dramatic increase in terminal galactose residues (i.e., Galβ1,4GlcNAc-) on myeloid cells. Thus, one can use the ST3Gal IV and/or ST3Gal I sialyltransferase as a detection moiety to determine whether ST3Gal IV and/or ST3Gal I is deficient in the cells.

In typical embodiments, the detection moieties are labeled with a detectable label. The detectable labels can be primary labels (where the label comprises an element that is detected directly or that produces a directly detectable element) or secondary labels (where the detected label binds to a primary label, as is common in immunological labeling). An introduction to labels, labeling procedures and detection of labels is found in Polak and Van Noorden (1997) Introduction to Immunocytochemistry, 2nd ed., Springer Verlag, NY and in Haugland (1996) Handbook of Fluorescent Probes and Research Chemicals, a combined handbook and catalogue published by Molecular Probes, Inc., Eugene, Oreg. Primary and secondary labels can include undetected elements as well as detected elements. Usefull primary and secondary labels in the present invention can include spectral labels such as fluorescent dyes (e.g., fluorescein and derivatives such as fluorescein isothiocyanate (FITC) and Oregon Green, rhodamine and derivatives (e.g., Texas red, tetrarhodimine isothiocynate (TRITC), etc.), digoxigenin, biotin, phycoerythrin, AMCA, CyDyes™, and the like), radiolabels (e.g., ³H, ¹²⁵I, ³⁵S, ¹⁴C, ³²P, ³³P, etc.), enzymes (e.g., horse radish peroxidase, alkaline phosphatase etc.), spectral calorimetric labels such as colloidal gold or colored glass or plastic (e.g., polystyrene, polypropylene, latex, etc.) beads. The label can be coupled directly or indirectly to a component of the detection assay (e.g., the detection reagent) according to methods well known in the art. As indicated above, a wide variety of labels can be used, with the choice of label depending on sensitivity required, ease of conjugation with the compound, stability requirements, available instrumentation, and disposal provisions.

Preferred labels include those that use: 1) chemiluminescence (using horseradish peroxidase or luciferase) with substrates that produce photons as breakdown products as described above) with kits being available, e.g., from Molecular Probes, Amersham, Boehringer-Mannheim, and Life Technologies/Gibco BRL; 2) color production. (using both horseradish peroxidase and/or alkaline phosphatase with substrates that produce a colored precipitate [kits available from Life Technologies/Gibco BRL, and Boehringer-Mannheim]); 3) hemifluorescence using, e.g., alkaline phosphatase and the substrate AttoPhos [Amersham] or other substrates that produce fluorescent products, 4) fluorescence (e.g., using Cy-5 [Amersham]), fluorescein, and other fluorescent tags]; 5) radioactivity. Other methods for labeling and detection will be readily apparent to one skilled in the art.

Preferred enzymes that can be conjugated to detection reagents of the invention include, e.g., luciferase, and horse radish peroxidase. The chemiluminescent substrate for luciferase is luciferin. Embodiments of alkaline phosphatase substrates include p-nitrophenyl phosphate (pNPP), which is detected with a spectrophotometer; 5-bromo-4-chloro-3-indolyl phosphate/nitro blue tetrazolium (BCIP/NBT) and fast red/napthol AS-TR phosphate, which are detected visually; and 4-methoxy-4-(3-phosphonophenyl) spiro[1,2-dioxetane-3,2′-adamantane], which is detected with a luminometer. Embodiments of horse radish peroxidase substrates include 2,2′azino-bis(3-ethylbenzthiazoline-6 sulfonic acid) (ABTS), 5-aminosalicylic acid (5AS), o-dianisidine, and o-phenylenediamine (OPD), which are detected with a spectrophotometer; and 3,3,5,5′-tetramethylbenzidine (TMB), 3,3′diaminobenzidine (DAB), 3-amino-9-ethylcarbazole (AEC), and 4-chloro-1-naphthol (4C1N), which are detected visually. Other suitable substrates are known to those skilled in the art.

In general, a detector which monitors a particular label is used to detect the label. Typical detectors include spectrophotometers, phototubes and photodiodes, microscopes, scintillation counters, cameras, film and the like, as well as combinations thereof. Examples of suitable detectors are widely available from a variety of commercial sources known to persons of skill. Commonly, an optical image of a substrate comprising bound labeling moieties is digitized for subsequent computer analysis.

Commercially available detection moieties that are suitable for use in the methods of the invention include SNA-fluorescein isothiocyanate (FITC) lectin (FL-1301, Vector Laboratories, Burlingame Calif.) and biotinylated SNA lectin (B-1305, Vector Laboratories) for α2,3 sialyl galactosides.

A reduction in ST3 Gal IV and/or ST3Gal I activity is evidenced by a substantial reduction in α2,3-sialylgalactosides in a sample obtained from the patient. Alternatively, methods for detecting levels of ST3Gal IV and/or ST3Gal I enzymatic activities can be used. As used herein, a “substantial reduction” in the appropriate sialylgalactoside levels or ST3Gal IV and/or ST3Gal I activity refers to a reduction of at least about 30% in the test sample compared to a non-immunodeficient control. Depending on the degree of reduction in inflammation desired, the reduction in ST3 Gal IV and/or ST3Gal I activity or α2,3-linked sialylgalactoside will be at least about 50%, more preferably at least about 75%, and most preferably sialylgalactoside or ST3Gal IV and/or ST3 Gal I levels will be reduced by at least about 90% in a sample from an animal that has a clotting disorder compared to a control. Again, however, monitoring of the extent of inflammation is the preferred method of monitoring the effectiveness of a treatment or prophylactic administration.

Transgenic Animals That Lack ST3 Gal IV Sialyltransferase

The invention also provides eukaryotic cells, as well as chimeric and transgenic nonhuman animals which contain cells, that lack at least one ST3Gal IV and/or at least one ST3Gal I gene that is typically found in wild-type cells of the animal. Methods for producing such cells and animals are also provided. These cells and animals are useful for several purposes, including the study of the mechanisms by which leukocyte extravasation and resulting inflammation occur. The “knockout” cells and animals can also be used for producing glycoproteins and glycolipids that, when produced in a wild-type cell or animal, would carry an α2,3-linked sialic acid residue that is not desirable for a particular application.

A “chimeric animal” includes some cells that lack the functional sialyltransferase gene of interest and other cells that do not have the inactivated gene. A “transgenic animal,” in contrast, is made up of cells that have all incorporated the specific modification which renders the sialyltransferase gene inactive. While a transgenic animal is capable of transmitting the inactivated sialyltransferase gene to its progeny, the ability of a chimeric animal to transmit the mutation depends upon whether the inactivated gene is present in the animal's germ cells.

The modifications that inactivate the sialyltransferase gene can include, for example, insertions, deletions, or substitutions of one or more nucleotides. The modifications can interfere with transcription of the gene itself, with translation and/or stability of the resulting mRNA, or can cause the gene to encode an inactive sialyltransferase polypeptide. For example, a mutation can be introduced into the promoter region of one or more ST3Gal IV and/or at least one ST3Gal I genes, in which case the gene is expressed at a reduced level, if at all. Alternatively, the coding region of the gene can be mutated.

The methods of the invention are useful for producing transgenic and chimeric animals of most vertebrate species. Such species include, but are not limited to, nonhuman mammals, including rodents such as mice and rats, rabbits, ovines such as sheep and goats, porcines such as pigs, and bovines such as cattle and buffalo. Methods of obtaining transgenic animals are described in, for example, Puhler, A., Ed., Genetic Engineering of Animals, VCH Publ., 1993; Murphy and Carter, Eds., Transgenesis Techniques: Principles and Protocols (Methods in Molecular Biology, Vol. 18), 1993; and Pinkert, C A, Ed., Transgenic Animal Technology: A Laboratory Handbook, Academic Press, 1994.

One method of obtaining a transgenic or chimeric animal having an inactivated ST3Gal IV and/or ST3Gal I gene in its genome is to contact fertilized oocytes with a vector that includes a ST3Gal IV- and/or ST3Gal I-encoding polynucleotide that is modified to contain an inactivating modification. For some animals, such as mice, fertilization is performed in vivo and fertilized ova are surgically removed. In other animals, particularly bovines, it is preferably to remove ova from live or slaughterhouse animals and fertilize the ova in vitro. See DeBoer et al., WO 91/08216. In vitro fertilization permits the modifications to be introduced into substantially synchronous cells. Fertilized oocytes are then cultured in vitro until a pre-implantation embryo is obtained containing about 16-150 cells. The 16-32 cell stage of an embryo is described as a morula. Pre-implantation embryos containing more than 32 cells are termed blastocysts. These embryos show the development of a blastocoel cavity, typically at the 64 cell stage. If desired, the presence of a desired inactivated ST3Gal IV gene in the embryo cells can be detected by methods known to those of skill in the art. Methods for culturing fertilized oocytes to the pre-implantation stage are described by Gordon et al. (1984) Methods Enzymol. 101: 414; Hogan et al. (1986) Manipulation of the Mouse Embryo: A Laboratory Manual, C. S. H. L. N.Y. (mouse embryo); Hammer et al. (1985) Nature 315: 680 (rabbit and porcine embryos); Gandolfi et al. (1987) J. Reprod. Fert. 81: 23-28; Rexroad et al. (1988) J. Anim. Sci. 66: 947-953 (ovine embryos) and Eyestone et al. (1989) J. Reprod. Fert. 85:715-720; Camous et al. (1984) J. Reprod. Fert. 72: 779-785; and Heyman et al. (1987) Theriogenology 27: 5968 (bovine embryos). Sometimes pre-implantation embryos are stored frozen for a period pending implantation. Pre-implantation embryos are transferred to an appropriate female resulting in the birth of a transgenic or chimeric animal depending upon the stage of development when the transgene is integrated. Chimeric mammals can be bred to form true germline transgenic animals.

Alternatively, the disrupted ST3Gal IV and/or ST3Gal I gene can be introduced into embryonic stem cells (ES). These cells are obtained from preimplantation embryos cultured in vitro. See, e.g., Hooper, M L, Embryonal Stem Cells: Introducing Planned Changes into the Animal Germline (Modern Genetics, v. 1), Int'l. Pub. Distrib., Inc., 1993; Bradley et al. (1984) Nature 309, 255-258. Transformed ES cells are combined with blastocysts from a nonhuman animal. The ES cells colonize the embryo and in some embryos form the germ line of the resulting chimeric animal. See, Jaenisch (1988) Science 240: 1468-1474. Alternatively, ES cells or somatic cells that can reconstitute an organism (“somatic repopulating cells”) can be used as a source of nuclei for transplantation into an enucleated fertilized oocyte giving rise to a transgenic mammal. See, e.g., Wilmut et al. (1997) Nature 385: 810-813.

The introduction of the modified ST3Gal IV and/or ST3Gal I gene into recipient cells can be accomplished by methods known to those of skill in the art. For example, the modified gene can be targeted to the wild type ST3Gal IV locus by homologous recombination. Alternatively, a recombinase system can be employed to delete all or a portion of a locus of interest. Examples of recombinase systems include, the cre/lox system of bacteriophage P1 (see, e.g., Gu et al. (1994) Science 265: 103-106; Terry et al. (1997) Transgenic Res. 6: 349-356) and the FLP/FRT site specific integration system (see, e.g., Dymecki (1996) Proc. Nat'l. Acad. Sci. USA 93: 6191-6196). In these systems, sites recognized by the particular recombinase are typically introduced into the genome at a position flanking the portion of the gene that is to be deleted Introduction of the recombinase into the cells then catalyzes recombination which deletes from the genome the polynucleotide sequence that is flanked by the recombination sites. If desired, one can obtain animals in which only certain cell types lack the sialyltransferase gene of interest. See, e.g., Tsien et al. (1996) Cell 87: 1317-26; Brocard et al. (1996) Proc. Nat'l. Acad. Sci. USA 93: 10887-10890; Wang et al. (1996) Proc. Nat'l. Acad. Sci. USA 93: 3932-6; Meyers et al. (1998) Nat. Genet. 18: 136-41).

### EXAMPLES

The following example is offered to illustrate, but not to limit the present invention. Knockout mice were constructed in which genes encoding the ST3Gal-IV sialyltransferase were disrupted. Studies of these mice demonstrated that ablation of this ST3Gal-IV sialyltransferase, which acts on both N- and O-glycans in vitro, results in a decrease in neutrophil recruitment and extravasation in response to an inflammatory stimulus in mice.

Materials and Methods

Gene Targeting of the ST3Gal-IV and Production of Mutant Mice

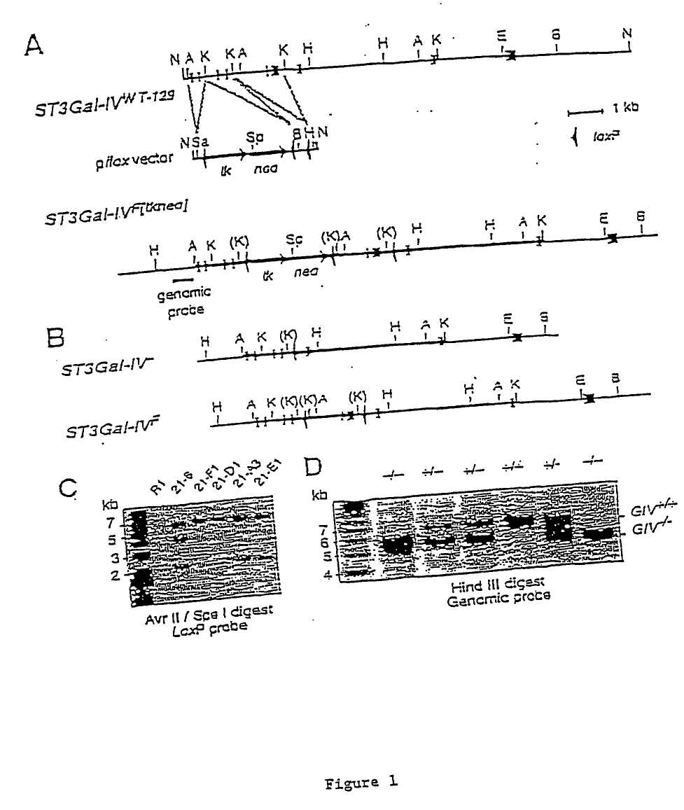

Isolation of mouse ST3Gal-IV genomic DNA and construction of a targeting vector bearing Cre loxP recombination signals was accomplished in a manner similar to that described by (Priatel et al. (1997) Glycobiology 7: 45-56). R1 ES cells (Nagy et al. (1993) Proc. Nat'l. Acad. Sci. USA 90: 8424-8) were electroporated with 10 μg of the linearized targeting construct shown in FIG. 1A, and the resulting clones were screened by Southern blotting using the genomic probe (FIG. 1A). Targeted ES cells were electroporated with 5 μg of Cre expression plasmid and subclones bearing the ST3Gal-IV^{-/-} and ST3Gal-IV^{F} alleles (FIG. 1B) were isolated. ST3Gal-IV^{-/-} and ST3Gal-IV^{F} chimeric mice were generated using standard techniques (Metzler et al. (1994) EMBO J. 13: 2056-65) and were crossed into the C57BL/6 background for the generation of heterozygous and homozygous offspring.

The ST3Gal-IV alleleic structure was analyzed by Southern blotting and PCR. The wild type ST3Gal-IV allele was detected using PCR primers adjacent to the deleted region (W5′: 5′-GAC GCC ATC CAC CTA TGA G (SEQ ID NO:1) and W3′: 5′-GGC TGC TCC CAT TCC ACT-3′ (SEQ ID NO:2)) resulting in a 260 bp fragment, while the mutant allele was detected using W5′ and a primer from the loxP region (M3′: 5′-GGC TCT TTG TGG GAC CAT CAG-3′ (SEQ ID NO:3)), yielding a 450 bp fragment.

Northern Blot Analysis

Total RNA from a panel of tissues obtained from a wild-type mouse, and from small intestine and colon of wild-type and ST3Gal-IV^{-/-} mice was isolated by cesium chloride density centrifugation. Five μg of total RNA was electrophoresed on a denaturing 1% agarose gel and transferred to nitrocellulose. Detection of the ST3Gal-IV message was accomplished by hybridizing to the labeled full-length ST3Gal-IV cDNA.

Flow Cytometry

Single cell suspensions of splenocytes were prepared and erythrocytes removed by ammonium chloride lysis. Cells were incubated in the presence of antibodies (below) in FACS buffer (2% FCS in PBS) for 20 minutes at 4° C. For E- or P-selectin binding, cells were treated wit 0.5 μg/ml of Fc Block (anti-CD32/16, PharMingen), then incubated with Gr-1 and either the E- or P-selectin-IgM chimera (Maly et al. (1996) Cell 86: 643-53) with or without addition of 5 mm EDTA for 30 minutes at 4° C. Cells were washed and incubated with a goat anti-human FITC conjugated secondary antibody (Sigma) as appropriate. Antibodies used were CD11a (M17/4), CD11b (M1/70), CD18 (C71/16), CD22 (Cy34.L), CD24 (M1/69), GD43 (S7 and 1B11), CD45 (30-F11), CD45R/B220 (RA3-6B2), CD62L (MEL-14), and Gr-1 (RB6-8C5) (PharMingen). The anti-PSGL-1 antibody, 4RA10 was a generous gift from Dr. D. Vestweber. Data were analyzed on a FACScan flow cytometer using CELL QUEST software (Becton Dickinson).

Hematology

Blood from the tail vein of methoxyfluorane anesthetized mice was collected into EDTA-coated polypropylene microtubes (Becton Dickinson). Analyses of red blood cells, white blood cells and platelet cell numbers and morphology were carried out manually and with a CELL-DYN 3500™ calibrated with normal mouse blood (UCSD Medical Center, Hillcrest).

Frozen sections of spleen or small intestine were fixed, permeabilized and blocked as previously described (Nichols, W. Cell 1998). The DBA-FITC lectin at 5 μg/ml and vWF antibody at 25 μg/ml were applied to the sections in PBS with 0.05% Tween 20, 0.05% triton X 100 and 5% goat serum and incubated overnight at 4° C. The slides were washed three times in PBS and a goat anti-rabbit rhodamine secondary (Jackson) was applied for 1 h. After three washes in PBS the slides were air dried and mounted with Gel/Mount (Biomeda, Foster City, Calif.). Bone marrow hematoxylin and eosin slides were prepared from cytospins of single cells suspensions.

Peritoneal Inflammation

Mice were injected intraperitoneally with 1 ml of 3% thioglycollate (Sigma). At the indicated times, mice were sacrificed and the peritoneal cavities lavaged with 10 ml of ice cold PBS containing 1% BSA and 0.5 mM EDTA. Red blood cells were removed by hypotonic lysis and leukocytes counted manually using a hemocytometer. Cytospins were stained with Leukostat (Sigma) and neutrophils counted. Peritoneal exudates were also stained with Gr-1 and F4/80 (Caltag) and analyzed by flow cytometry.

Statistical Analysis

Data were analyzed by ANOVA and Scheffe's t test for unpaired samples using StatView® software.

Results

Disruption of the ST3Gal-IV Gene by Targeted Mutagenesis

The ST3Gal-IV sialyltransferase is a type II Golgi enzyme that belongs to a family of six conserved members. A mouse genomic clone encompassing the twelve exon protein-coding region of the gene was used in constructing a gene-targeting vector designed to control exon deletion by Cre-loxP recombination (FIG. 1A). Homologous recombination of the targeting vector in embryonic stem (ES) cells incorporated selection markers and 3 loxP sites for the subsequent production of systemic ST3Gal-IV^{-/-} or conditional ST3Gal-IV^{F} mutations in vivo (FIGS. 1B and 1C). These alleles were transmitted into the mouse germline and offspring homozygous for either the ST3Gal-IV^{-/-} or ST3Gal-IV^{F} allele were generated. Such offspring, which occurred at a frequency of 25% of littermates, lacked overt physical or behavioral abnormalities, developed normally and were fully fertile. Mice homozygous for ST3Gal-IV^{-/-} allele were further analyzed.

ST3Gal-IV mRNA Levels and Terminal Sialic Acid Production

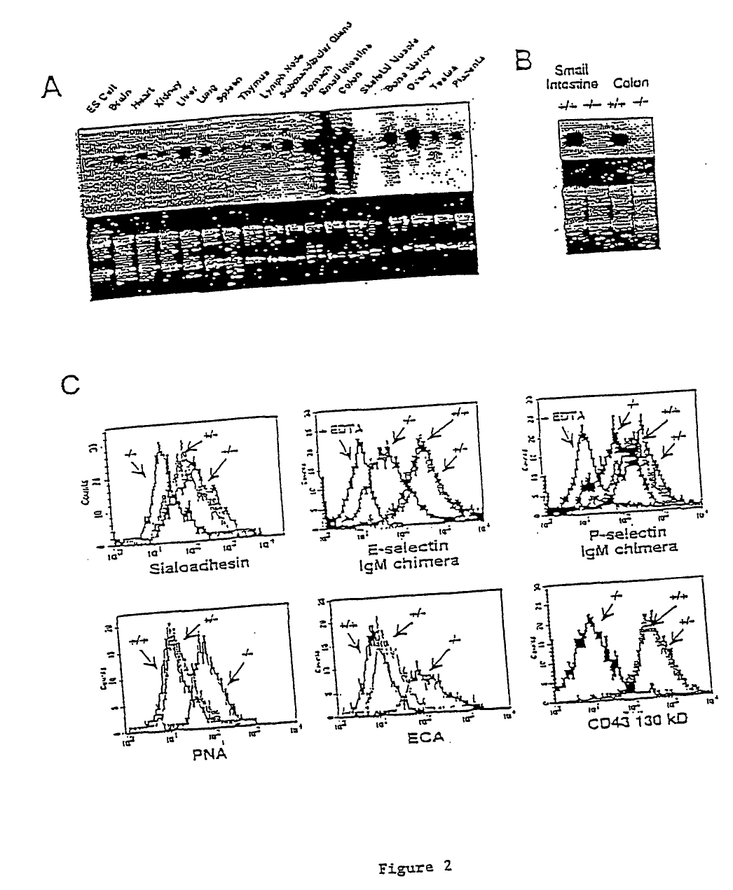

ST3Gal-IV mRNA as detected by Northern blotting is broadly expressed in mouse tissues and is highly expressed in the gastrointestinal tract (FIG. 2A). ST3Gal-1-mice show a loss of mRNA from the small intestine and colon, suggesting that in these tissues the mRNA formed is unstable (FIG. 2B).

Several members of the siglec (sialoadhesin) family of lectins bind α2,3 sialic acids, although specific counterreceptors have not been defined (Crocker et al. (1997) Glycoconj. J. 14: 601-9). Myeloid cells of homozygous null (−/−) mice exhibited abrogation of siglec 1 binding (FIG. 2C)). This suggested that the ST3Gal-IV sialyltransferase is a key component of the binding site for this lectin, which has been proposed to be involved in myeloid cell function. Also observed was a significant loss of E-selectin binding and aminor reduction in P-selectin binding FIG. 2C), which indicate that ST3Gal-IV is involved in selectin ligand formation, although other α2,3 sialyltransferases may be compensating for the majority of this activity.

Lymph node development and cellularity was normal as determined by histologic analysis, FACS and cell counting, and therefore L-selectin binding was not assessed in these mice.

An increase was observed in binding of peanut agglutinin (PNA) and Erythrina cristagalli (ECA) lectin binding, which recognize Galβ1,3GalNAc and Galβ1,4GlcNAc respectively, to myeloid cells from the bone marrow and spleen. This indicates an increase in exposure of terminal galactose, thus confirming the loss of a subset of terminal α2,3 sialic acids from cell surface glycoproteins on cells of the myeloid lineage (FIG. 2C). Interestingly, an epitope normally found on core 2 O-glycans of the cell surface adhesion molecule CD43, which is recognized by the 1B11 mAb, was absent in ST3Gal-IV^{-/-} mice.

ST3Gal-IV Deficiency Results in Loss of Neutrophil Recruitment

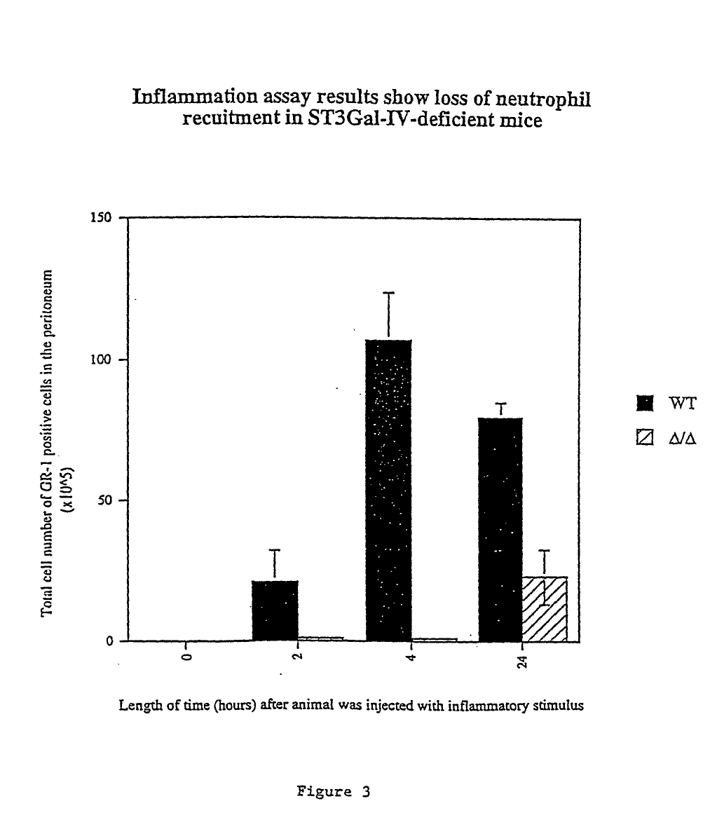

FIG. 3 shows that Gr-1⁺ neutrophil recruitment does not occur in response to an inflammatory stimulus in animals that lack ST3Gal-IV. At 2 and 4 hours post-stimulus, the decrease in neutrophil recruitment was essentially complete. At 24 hours, some recruitment of Gr-1⁺ cells was observed; these represented macrophages.

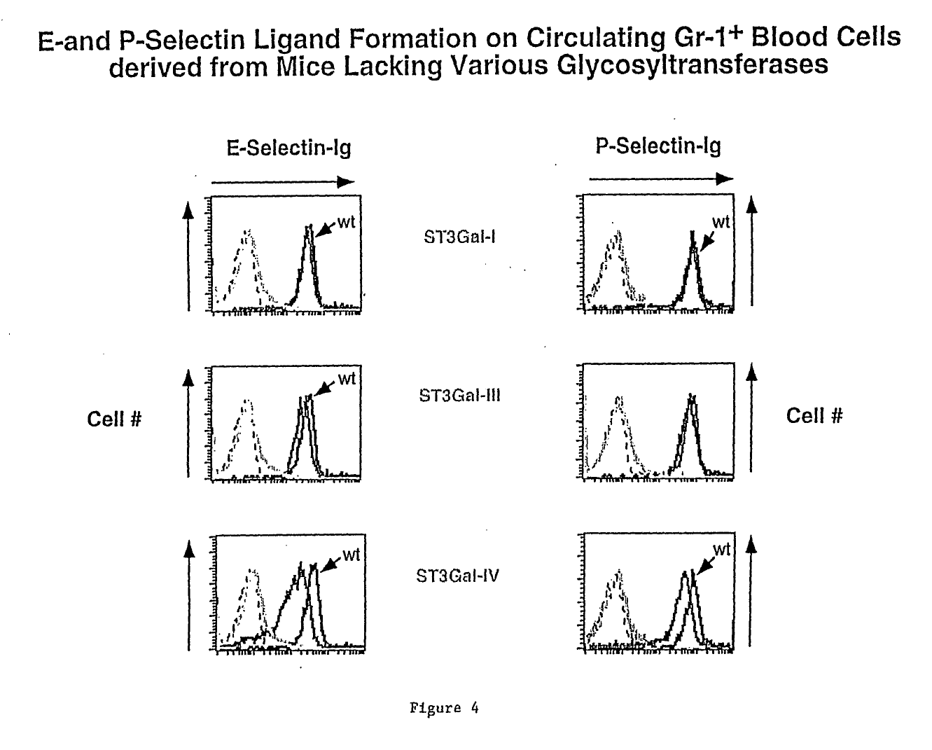

Gr-1⁺ cells from ST3Gal-IV-deficient animals exhibited a partial reduction in E- and P-selectin binding (FIG. 4). The magnitude of the reduction in selectin binding was relatively minimal compared to that observed with cells from knockout mice that lack a Core 2 GlcNAc transferase (light line in FIG. 4). Core 2 GlcNAc transferase-deficient mice are described in PCT application WO00/31109.

Discussion

Absence or inhibition of the ST3Gal-IV sialyltransferase resulted in a complete loss of neutrophil recruitment and extravasation following challenge with an inflammatory stimulus. Only minor decreases were observed in selectin ligand formation. This indicates that the effect of ST3Gal-IV sialyltransferase deficiency is unique from selectin involvement Moreover, no leukocytosis occurred with loss of ST3Gal-IV sialyltransferase. These results indicate that the ST3Gal-1 sialyltransferase is involved in a process different from, and probably downstream from, selectin binding and ICAM-1 involvement. For example, extravasation may be defective due to deficient cell signalling involving blood or endothelial cell types.

### Example 2

Construction and Analysis of Transgenic Mice Deficient in Sialyltransferase ST3Gal I

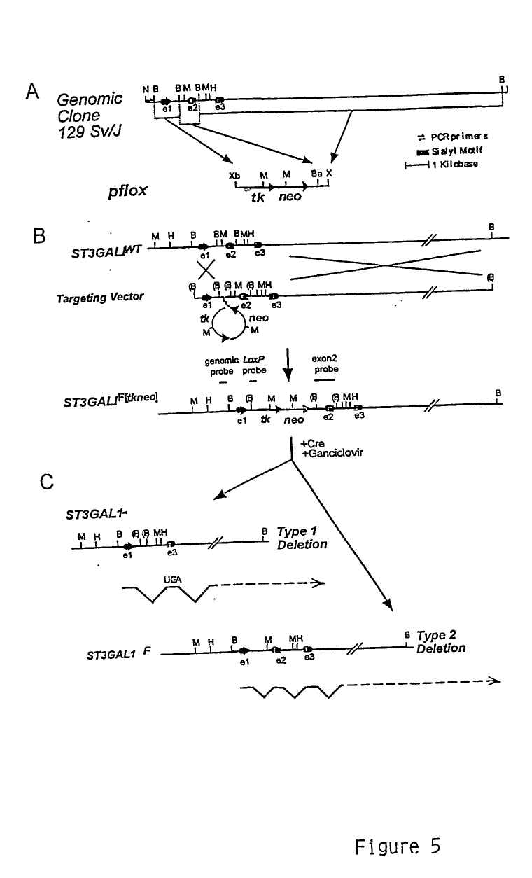

Production of ST3Gal I deficient mice was accomplished similarly to production of ST6Gal deficiency in mice as described in Example 1. The essential difference was the location of the mutation (i.e., the ST3Gal I locus and not the ST6Gal locus). Mutation of the ST3Gal I allele involved deletion of an exon that is essential for ST3Gal I enzyme production (see FIG. 5). While any manner of insertional mutagenesis would also produce the same end result (i.e., ST3Gal I deficiency), we chose to mutate the gene by Cre recombination with deletion of exon 2. Placement of loxP sites in genomic context and surrounding exon 2 is shown in FIG. 5A. In FIG. 5B, the modified ST3Gal I allele is depicted as it occurred in embryonic stem cells following homologous recombination. Subsequently, (FIG. 5C) Cre recombination of ES cells heterozygous for the F^{[tkneo]} allele, followed by ganciclovir selection, provided the type 1 and type 2 deletions. ES cells harboring the type 1 deletion were used to produce mice lacking ST3Gal I function.

Mice lacking a wild-type ST3Gal I allele developed normally and appeared grossly unaltered in a pathogen-free environment. However, as a result of ST3Gal I deficiency, these mice had lost the vast majority of their ability to recruit leukocytes to a site of inflammatory stimulus. This reduction in leukocyte recruitment was not due to a reduction in selectin binding ability.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

What is claimed is:

1. A method for reducing or preventing inflammation in a mammal, the method comprising administering to the mammal an agent that causes a decrease in the amount of cell-surface oligosaccharides that comprise a terminal α2,3-linked sialic acid, wherein the cell-surface oligosaccharides are attached to a cell involved in inflammation.

2. The method of claim 1, wherein the sialylated oligosaccharide comprises Siaα2,3Galβ1-3GalNAc.

3. The method of claim 1, wherein the agent inhibits expression of a gene encoding a glycosyltransferase involved in synthesis of the sialylated oligosaccharide.

4. The method of claim 3, wherein the glycosyltransferase is an α2,3 sialyltransferase.

5. The method of claim 4, wherein the sialyltransferase is selected from the group consisting of ST3Gal-IV and ST3Gal-I.

6. The method of claim 5, wherein the sialyltransferase is ST3Gal-IV.

7. The method of claim 4, wherein the agent decreases expression of a gene that encodes the sialyltransferase.

8. The method of claim 7, wherein the agent is an antisense nucleic acid that hybridizes to an ST3Gal-IV- or ST3Gal-1-encoding nucleic acid.

9. The method of claim 1, wherein the agent inhibits enzymatic activity of a glycosyltransferase polypeptide involved in synthesis of the sialylated oligosaccharide.

10. The method of claim 9, wherein the glycosyltransferase is an α2,3 sialyltransferase.

11. The method of claim 10, wherein the sialyltransferase is selected from the group consisting of ST3Gal-IV and ST3Gal-I.

12. The method according to claim 10, wherein the agent comprises an analog of a sialic acid precursor.

13. The method according to claim 9, wherein the agent comprises an analog of a donor substrate, or an analog of an acceptor substrate, for the glycosyltransferase.

14. The method according to claim 13, wherein the agent comprises an analog of a sugar nucleotide.

15. The method of claim 1, wherein the agent comprises a sialidase which cleaves sialic acid from an the sialylated oligosaccharide.

16. The method of claim 1, wherein the agent is administered in conjunction with administration of a drug for which inflammation is a potential side effect.

17. The method of claim 16, wherein the agent is administered before or simultaneously with the drug for which inflammation is a potential side effect.

18. The method of claim 1, wherein the method is performed as a prophylactic measure against inflammation.

19. The method of claim 1, wherein the method is performed as a therapeutic measure against inflammation.

20. The method of claim 1, wherein the cells involved in inflammation are neutrophils.

21. The method of claim 1, wherein the method does not significantly decrease neutrophil binding to selecting.

22. The method of claim 1, wherein the decrease in the amount of cell-surface oligosaccharides that comprise a terminal α2,3-linked sialic acid results from an agent-mediated decrease in acceptor moieties for an α2,3-sialyltransferase.

23. The method of claim 22, wherein the agent comprises a glycosyltransferase which converts an acceptor substrate for a sialyltransferase to an oligosaccharide which is not a sialyltransferase acceptor substrate.

24. The method of claim 23, wherein the glycosyltransferase is a fucosyltransferase.

25. A method of inhibiting leukocyte extravasation in a mammal, the method comprising administering to the mammal an agent that causes a decrease in the amount of α2,3 sialylated oligosaccharides attached to cells involved in inflammation.


SEQUENCE LISTING (3 sequences)

SEQ ID NO: 1 - DNA, 19 bp, Artificial Sequence
  Description of Artificial SequenceW5′ PCR primer adjacent to deleted region in wild type ST3Gal-IV allele
gacgccatccacctatgag

SEQ ID NO: 2 - DNA, 18 bp, Artificial Sequence
  Description of Artificial SequenceW3′ PCR primer adjacent to deleted region in wild type ST3Gal-IV allele
ggctgctcccattccact

SEQ ID NO: 3 - DNA, 21 bp, Artificial Sequence
  Description of Artificial SequenceM3′ PCR primer from loxP region in mutant ST3Gal-IV allele
ggctctttgtgggaccatcag